(19) United States
(12) Patent Application Publication
(10) Pub. No.: US 2021/0293750 A1
(43) Pub. Date: Sep. 23, 2021

(54) BIOMOLECULE ANALYSIS DEVICE

(71) Applicant: HITACHI HIGH-TECH CORPORATION, Tokyo (JP)

(72) Inventors: Rena AKAHORI, Tokyo (JP); Yusuke GOTO, Tokyo (JP); Itaru YANAGI, Tokyo (JP); Mayu AOKI, Tokyo (JP)

(73) Assignee: HITACHI HIGH-TECH CORPORATION, Tokyo (JP)

(21) Appl. No.: 17/265,834

(22) PCT Filed: Jul. 3, 2019
(86) PCT No.: PCT/JP2019/026406

§ 371 Date: Feb. 4, 2021

(30) Foreign Application Priority Data
Aug. 28, 2018 (JP) 2018-159481

(51) Int. Cl. G01N 27/447; G01N 33/487

(52) U.S. Cl. CPC G01N 27/44791; G01N 33/48721

(57) ABSTRACT

A biomolecule analysis device includes a thin film having a nanopore, a liquid tank that is disposed in contact with the thin film and contains an electrolyte solution, an electrode in contact with the liquid tank, a measurement device connected to the electrode, and a controller that controls a voltage to be applied to the electrode, in accordance with a measurement result of the measurement device. A biomolecule is introduced into the electrolyte solution. A control strand and a molecular motor are connected to a first end portion of the biomolecule, and the control strand is bound to a primer on an upstream of the control strand and has a spacer on a downstream of the control strand.

### TECHNICAL FIELD

The present invention relates to an apparatus for analyzing a biomolecule (biopolymer).

### BACKGROUND ART

In the field of next-generation DNA sequencers, a method of electrically and directly measuring the base sequence of a biomolecule (referred to as “DNA” below) without carrying out an extension reaction or a fluorescent labeling has attracted attention. Specifically, research and development of nanopore DNA sequencing methods are being actively promoted. This method is a method of directly measuring a DNA strand without using a reagent, and determining a base sequence.

In this nanopore DNA sequencing method, the base sequence is measured by measuring the blockade current generated by a DNA strand passing through a pore (referred to as a “nanopore” below) formed in a thin film. That is, since the blockade current changes depending on the difference between the individual base types contained in the DNA strand, the base types can be sequentially identified by measuring the amount of the blockade current. In this method, a template DNA is not amplified by an enzyme, and a labeling substance such as a phosphor is not used. Therefore, this method has high throughput and low running cost, and enables long-base DNA deciphering.

A device for biomolecular analysis used when analyzing DNA in the nanopore DNA sequencing method generally includes first and second liquid tanks filled with an electrolyte solution, a thin film for partitioning the first and second liquid tanks, and first and second electrodes respectively provided in the first and second liquid tanks. The device for biomolecular analysis can also be configured as an array device. The array device is a device including a plurality of sets of liquid chambers partitioned by a thin film. For example, the first liquid tank is used as a common tank, and the second liquid tank is used for a plurality of individual tanks. In this case, electrodes are disposed in the common tank and each of the individual tanks.

In this configuration, a voltage is applied between the first liquid tank and the second liquid tank, and an ionic current corresponding to the diameter of the nanopore flows through the nanopore. Further, a potential gradient is generated in the nanopore in accordance with the applied voltage. When the biomolecule is introduced into the first liquid tank, the biomolecule is transported to the second liquid tank via the nanopore in response to the diffusion and the generated potential gradient. At this time, biomolecular analysis is performed in accordance with the blockade rate of each nucleic acid that blocks the nanopore. Note that, the biomolecule analysis device includes a measurement device that measures the ionic current (blockade signal) flowing between the electrodes provided in the device for biomolecular analysis, and acquires sequence information of biomolecules based on the value obtained by measuring the ionic current (blockade signal).

As one of objects of the nanopore DNA sequencing method, transport control of DNA passing through the nanopore is exemplified. It is considered as follows: in order to measure the difference between individual base types contained in a DNA strand by the amount of the blockade current, the passing speed of DNA through the nanopore needs to be 100 μs or more per base, from the current noise and the time constant of fluctuation of DNA molecules during measurement. However, the passing speed of DNA through the nanopore is usually as fast as 1 μs or less per base, and it is difficult to sufficiently measure the blockade current derived from each base.

As one of transport control methods, there is a method of realizing the transport control of DNA passing through the nanopore by using the force to transport and control the single strand that serves as a template when DNA polymerase carries out a synthetic reaction or when helicase breaks a single strand of double-stranded DNA (see NPL 1). When the polymerase binds to the primer annealed DNA which passes through the nanopore by electrophoresis and the synthesis reaction of the polymerase starts, the polymerase pulls the DNA in the opposite direction against the direction of electrophoresis. At this time, the measurement device may acquire the ionic current signal corresponding to the base type.

### CITATION LIST

### Non-Patent Literature

NPL 1: Gerald M Cherf et al., Nat. Biotechnol. 2012

### SUMMARY OF INVENTION

### Technical Problem

On the other hand, in this transport control method, it is required to precisely control a synthesis start point. The polymerase searches for the boundary between single and double strands and starts synthesis. In this case, a mechanism of realizing the synthesis only in the vicinity of the nanopore and not causing the synthesis in an electrolyte solution (reaction solution) away from the vicinity of the nanopore is required. However, in the conventional device, there is a problem that the synthesis by the polymerase is started in the electrolyte solution away from the nanopore, and thus it is not possible to precisely perform the biomolecule analysis.

### Solution to Problem

In order to solve the above problems, according to the present invention, a biomolecule analysis device includes a thin film having a nanopore, a liquid tank that is disposed in contact with the thin film and contains an electrolyte solution, an electrode in contact with the liquid tank, a measurement device connected to the electrode, and a controller that controls a voltage to be applied to the electrode, in accordance with a measurement result of the measurement device. A biomolecule is introduced into the electrolyte solution. A control strand and a molecular motor are connected to a first end portion of the biomolecule, and the control strand is bound to a primer on an upstream of the control strand and has a spacer on a downstream of the control strand.

Further, according to the present invention, there is provided a biomolecule analysis method for analyzing a biomolecule, the method includes introducing the biomolecule into a liquid tank, the biomolecule having a first end portion connected to a control strand and a molecular motor, the control strand being bound to a primer on an upstream and having a spacer on a downstream, the liquid tank being disposed in contact with a thin film and containing an electrolyte solution, and the thin film having a nanopore, applying a voltage to the liquid tank and introducing the biomolecule into the nanopore, bringing the primer into contact with the molecular motor in the biomolecule introduced into the nanopore, transporting the biomolecule in the nanopore by a synthetic reaction of the biomolecule after contact between the primer and the molecular motor, and measuring a change of a current flowing in the nanopore during the transport.

### Advantageous Effects of Invention

According to the present invention, it is possible to precisely control a synthesis start point while performing transport control of a biomolecule.

### BRIEF DESCRIPTION OF DRAWINGS

FIG. 1 is a schematic diagram illustrating an outline of a configuration of a biomolecule analysis device according to a first embodiment.

FIG. 2 is a schematic diagram illustrating an effect of a spacer 113.

FIG. 3A is a conceptual diagram illustrating an operation of a control strand 111 and a molecular motor 110.

FIG. 3B is a flowchart illustrating a procedure for measuring a biomolecule 109 in the first embodiment.

FIG. 4 illustrates a result of an experiment on the effect of the spacer 113.

FIG. 5 illustrates experimental data on an effect of the molecular motor 110.

FIG. 6 illustrates a result obtained by measuring a blockade current for a biomolecule (model sample sequences) in which only adenine (A) and cytosine (C) are repeatedly formed by one base at a time (for example, 50 each), as a template biomolecule.

FIG. 7 is a schematic diagram illustrating an example of a configuration of another biomolecule analysis device 700.

FIG. 8 is a flowchart illustrating a measurement procedure by the biomolecule measuring devices 100 and 700.

FIG. 9A is a schematic diagram illustrating a structure of the biomolecule 109 to be measured in a biomolecule measuring device according to a second embodiment.

FIG. 9B is a schematic diagram illustrating an outline of measurement of the biomolecule 109 in the second embodiment.

FIG. 9C is a schematic diagram illustrating the outline of the measurement of the biomolecule 109 in the second embodiment.

FIG. 10A is a schematic diagram illustrating a structure of the biomolecule 109 to be measured in a biomolecule measuring device according to a third embodiment.

FIG. 10B is a schematic diagram illustrating an outline of measurement of the biomolecule 109 in the third embodiment.

FIG. 10C is a schematic diagram illustrating the outline of the measurement of the biomolecule 109 in the third embodiment.

FIG. 10D is a schematic diagram illustrating the outline of the measurement of the biomolecule 109 in the third embodiment.

FIG. 11A is a schematic diagram illustrating a structure of the biomolecule 109 to be measured in a biomolecule measuring device according to a fourth embodiment.

FIG. 11B is a schematic diagram illustrating an outline of measurement of the biomolecule 109 in the fourth embodiment.

FIG. 11C is a schematic diagram illustrating the outline of the measurement of the biomolecule 109 in the fourth embodiment.

FIGS. 12A through 12F is a schematic diagram illustrating an outline of measurement of the biomolecule 109 in a fifth embodiment.

FIG. 13 is a timing chart illustrating waveforms of an applied voltage and a signal in the measurement of the biomolecule 109 in the fifth embodiment.

### DESCRIPTION OF EMBODIMENTS

Hereinafter, embodiments of the present invention will be described with reference to the drawings. Although the accompanying drawings show specific examples in accordance with the principles of the present invention, the examples are for the purpose of understanding the present invention and are not used for a limited interpretation of the present invention.

### First Embodiment

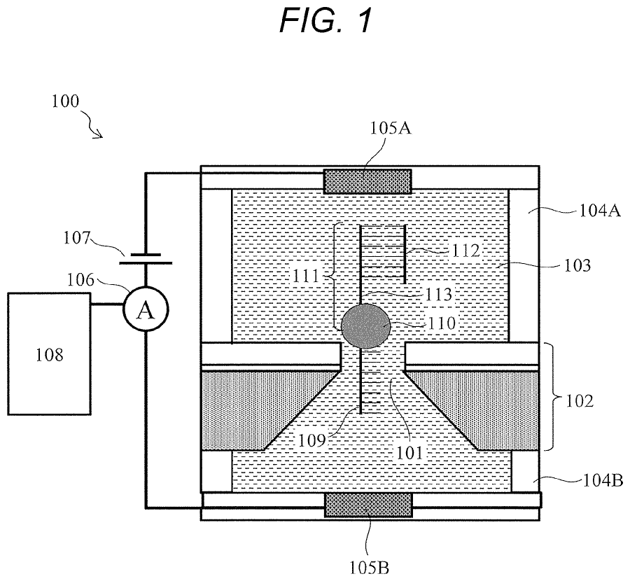

An outline of a configuration of a biomolecule analysis device according to a first embodiment will be described with reference to FIG. 1. The device is a device for biomolecular analysis that measures an ionic current by a blockade current method. The biomolecule analysis device includes a thin film 102 on which a nanopore 101 is formed, a pair of liquid tanks 104 (first liquid tank 104A and a second liquid tank 104B) that disposed to interpose the thin film 102 therebetween and be in contact with the thin film 102 and that are filled with an electrolyte solution 103, and a pair of electrodes 105 (first electrode 105A and second electrode 105B) that are respectively in contact with the first liquid tank 104A and the second liquid tank 104B. In measurement, a predetermined voltage from a voltage source 107 is applied between the pair of electrodes 105, and thus a current flows between the pair of electrodes 105. The magnitude of the current flowing between the electrodes 105 is measured by an ammeter 106, and the measured value is analyzed by a computer 108.

For example, KCl, NaCl, LiCl, and CsCl are used for the electrolyte solution 103. Urea of 4M or more, DMSO, DMF, and NaOH can be mixed in the solution in the second liquid tank 104B into which a molecular motor described later is not introduced, in order to suppress the formation of self-complementary strands of biomolecules. Further, it is possible to mix a buffer to stabilize the biomolecule. As the buffer, Tris, EDTA, PBS, and the like are used. The first electrode 105A and the second electrode 105B may be made of, for example, Ag, AgCl, or Pt.

A biomolecule (DNA strand or the like) 109 as a measurement target is introduced into the electrolyte solution 103. The biomolecule 109 includes, for example, a molecular motor 110 and a control strand 111 at one end thereof. The molecular motor is formed by a polymerase. Further, the control strand 111 is bound to a primer 112 at one end on a side farther from the molecular motor 110, and the control strand 111 has a spacer 113 at one end on a side closer to the molecular motor 110. Since the spacer 113 is provided, the primer 112 is not in contact with the molecular motor 110, and a synthetic reaction does not proceed until the biomolecule 109 reaches the nanopore 101. When the molecular motor 110 reaches the nanopore 101, and thus the control strand 111 is deformed, and the primer 112 comes into contact with the molecular motor 110, the synthetic reaction is started for the first time. Therefore, with the above structure, it is possible to control a synthesis start timing of the molecular motor 110 and to improve the measurement yield. This point will be described in detail later.

Note that, in the device illustrated in FIG. 1, one thin film 102 has only one nanopore 101, but this is just an example. The device may be set to an array device configured in a manner that a plurality of nanopores 101 is formed in the thin film 102, and the respective regions of the plurality of nanopores 101 are separated by partition walls. In the array device, the first liquid tank can be used as a common tank, and the second liquid tank can be used as a plurality of individual tanks. In this case, electrodes can be arranged in the common tank and the individual tanks, respectively.

The biomolecule analysis device in FIG. 1 includes a measurement device and a computer 108 as a controller. The measurement device measures an ionic current (blockade signal) flowing between the electrodes. The controller controls a voltage to be applied to the first electrode 105A and the second electrode 105B based on the measurement result. The computer 108 acquires sequence information of the biomolecule 109 based on the value of the measured ionic current (blockade signal). On the other hand, since the electrode is provided inside the nanopore 101, it is also possible to acquire the information on the biomolecule 109 by acquiring a tunnel current or detecting a change in transistor characteristics.

Here, the biomolecule 109 to be measured, the control strand 111, the primer 112, and the spacer 113 will be described in more detail.

In the device in FIG. 1, when a voltage is applied between the first electrode 105A and the second electrode 105B, a potential difference occurs between both sides of the thin film 102 on which the nanopore 101 is formed. Then, the biomolecule 109 dissolved in the upper liquid tank 104A is electrophoresed in a direction of the liquid tank 104B located on the lower side. The ammeter 106 includes an amplifier and an ADC (Analog to Digital Converter). The amplifier amplifies the current flowing between the electrodes by applying a voltage (not illustrated). The detected value being an output of the ADC is output to the computer 108. The computer 108 collects and records the detected current value.

The control strand 111 to be bound to the biomolecule 109 is provided separately and is introduced into the liquid tank 104A after pretreatment for sample preparation. A buffer suitable for driving the molecular motor 110 is allowed to coexist in the electrolyte of the liquid tank 104A. A buffer suitable for the molecular motor to be used is used as the buffer, and (NH4) 2SO4, KCl, MgSO4, Tween, Tris-HCl and the like are generally mixed.

In the biomolecule analysis device, it is necessary to perform transport control of the biomolecule when the biomolecule passes through the nanopore. The transport control in the device of the first embodiment is mainly performed by the molecular motor 110. The transport control by the molecular motor 110 needs to be started only in the vicinity of the nanopore 101. As a result of close examination by the inventor, the following were found. That is, if the control strand 111 is bound to the biomolecule 109 to be read, and the spacer 113 is provided on the molecular motor 110 side of the control strand 111, it is possible to start the transport control by the molecular motor 110 only when the biomolecule 109 reaches the vicinity of the nanopore 101.

In this case, the control strand 111 preferably satisfies the following conditions (a) to (d).

(a) The control strand 111 exists on the upstream of the biomolecule 109 (on the opposite side of the molecular motor 110 from the biomolecule to be measured).

(b) The primer 112 is bound to the upstream (side far from the molecular motor 110) of the control strand 111. That is, the far side of the control strand 111 is set to a primer binding site.

(c) The spacer 113 is provided on the downstream of the control strand 111.

(d) The length of the spacer 113 can be 2 mer or more as an example.

In the electrolyte solution 103, when a voltage is applied from the voltage source 107 to the upstream side and the downstream side of the nanopore 101 through the first electrode 105A and the second electrode 105B, an electric field is generated in the vicinity of the nanopore 101. The force of the electric field causes the biomolecule 109 to be introduced into the nanopore 101 and then pass through the nanopore 101. On the other hand, since the dimension Dm of the molecular motor 110 is larger than the diameter Dn of the nanopore 101, it is not possible for the molecular motor 110 to pass through the nanopore 101. At this time, the synthesis reaction is started when the primer 112 in the control strand 111 approaches the molecular motor 110 that stays in the vicinity of the nanopore 101.

As a result, the biomolecule 109 is pulled up from the nanopore 101 to the upstream side (first electrode 105A side) by the force when the molecular motor 110 extends the complementary strand. Then, the biomolecule 109 composed of nucleic acid is analyzed from the change in a signal acquired at this time. The result of such analysis is useful in fields such as testing, diagnosis, treatment, drug discovery, and basic research. In practice, when the biomolecule 109 prepared as described above is subjected to a synthetic reaction in an electrolyte solution without the nanopore 101, no synthetic reaction occurs. A signal which is derived from molecule transport and is obtained by the biomolecule passing is confirmed when the current is measured using the thin film 102 having the nanopore 101.

A point that configuration of the control strand 111 contributes to controlling the synthesis start will be specifically described below. In the device in FIG. 1, since the biomolecule 109 has not reached the nanopore 101 at the beginning of application of the voltage, a current corresponding to the diameter Dn of the nanopore 101 is measured by the ammeter 106. Then, the biomolecule 109 is introduced into the liquid tank 104A and reaches the vicinity of the nanopore 101 by an electric field.

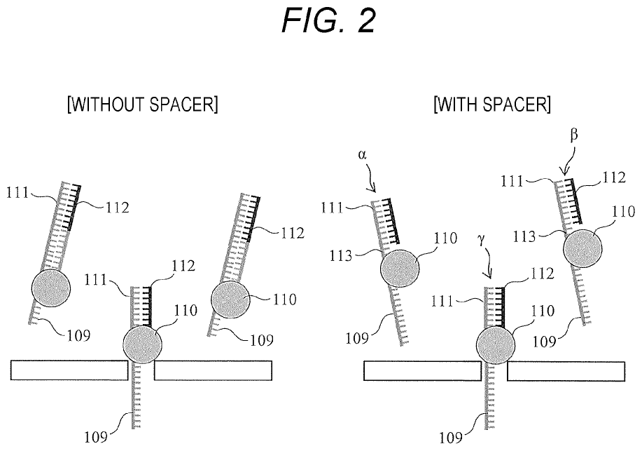

A problem occurring when the spacer 113 is not provided in the control strand 111 bound to the biomolecule 109 will be described with reference to FIG. 2. As illustrated in FIG. 2, when the spacer 113 is not provided, and the primer 112 is in contact with the molecular motor 110, the molecular motor 110 starts a synthetic reaction (extension reaction) from the primer 112 before the biomolecule 109 is introduced into the nanopore 101. If the synthetic reaction is completed before the biomolecule 109 is introduced into the nanopore 101, it is not possible for the biomolecule 109 to pass through the nanopore 101. Alternatively, as the extension reaction proceeds, the length (analysis length) of the biomolecule 109, which allows measurement of this device by passing through the nanopore 101 becomes shorter. The nanopore method has an advantage that measurement is possible with an analysis length larger than that of the biomolecule 109 as compared with other methods. However, if the extension reaction proceeds as described above, the advantage is lost.

As one of the measures to eliminate such yield reduction factors, it may be possible to take measures to eliminate them by reaction control with a block oligomer (G. M. Cherf et. Al., Nat. Biotechnol. (2012)). However, the use of two types of molecules, a primer and a block oligomer, requires the two types of molecules perform binding at the same time. Thus, as long as the binding occurs in a stochastic process, this is one of the factors for lowering the yield. In order to eliminate all causes of occurrence of the yield reduction, it is necessary that a mechanism for stopping the synthetic reaction is incorporated in advance and that a plurality of stochastic processes are not provided.

As the solution of the above problem, in the first embodiment, the primer 112 is provided on the upstream side (side far from the molecular motor 110) of the control strand 111, and the spacer 113 is provided on the downstream side (side close to the molecular motor 110). The termination of the primer 112 and the molecular motor 110 are separated by the spacer 113. Thus, the synthesis reaction is not started before the molecular motor 110 reaches the nanopore 101 (reference signs α and β in FIG. 2). When the biomolecule 109 passes through the nanopore 101, and then the molecular motor 110 reaches the nanopore 101, and thus the molecular motor 110 and one end of the primer 112 come into contact with each other, the synthesis reaction and transport control can be started (reference sign γ in FIG. 2).

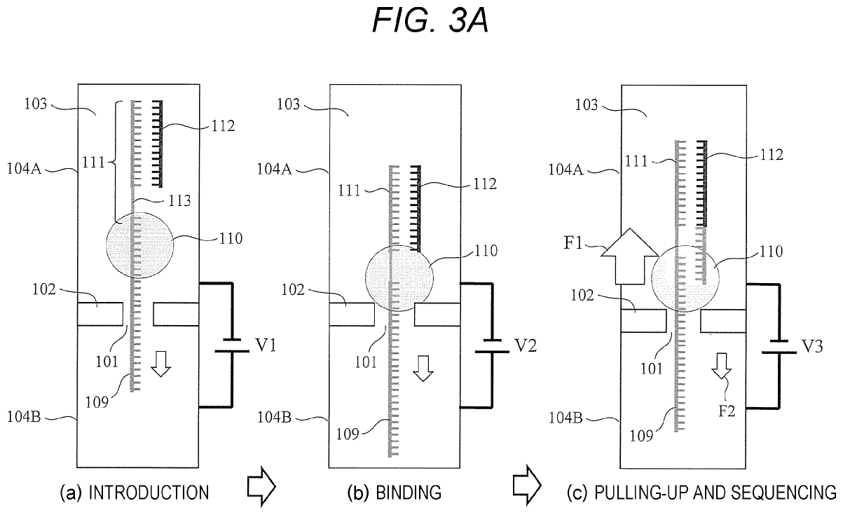

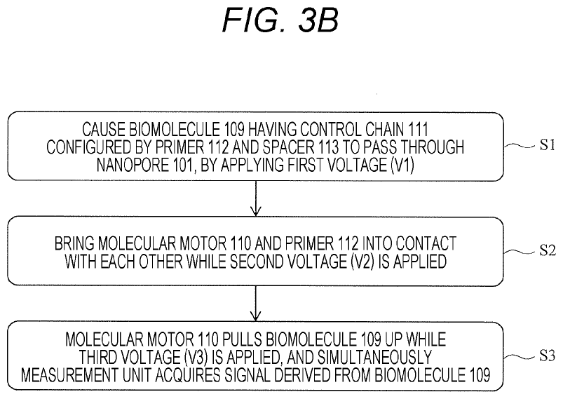

The operation of the control strand 111 and the analysis method of the biomolecule will be described in more detail with reference to FIGS. 3A and 3B. FIG. 3A is a conceptual diagram illustrating the operation of the control strand 111 and the molecular motor 110. FIG. 3B is a flowchart illustrating a procedure for measuring the biomolecule 109 in the first embodiment. As schematically illustrated in FIG. 3A, the biomolecule 109 is introduced into the liquid tank 104A containing the electrolyte solution 103, in a state where the control strand 111 is bound to the biomolecule 109. The molecular motor 110 is dissolved in the electrolyte solution 103. The biomolecule 109 and the molecular motor 110 are bound to the control strand 111 in the electrolyte solution 103. Note that, it is also possible to bind the control strand 111 or the molecular motor 110 to the biomolecule 109 in another solution and then introduce the biomolecule into the liquid tank 104A.

The control strand 111 is bound to the primer 112 on the upstream and has the spacer 113 on the downstream of the control strand. The primer 112 and the molecular motor 110 are separated by the spacer 113. As described above, a first voltage V1 is applied, through the first electrode 105A and the second electrode 105B, to the liquid tanks 104A and 104B in which the biomolecule 109 having the control strand 111 composed of the primer 112 and the spacer 113 is dissolved in the electrolyte solution 103 (Step S1 in FIG. 3B). Thus, the biomolecule 109 to which the molecular motor 110 is bound is introduced into the nanopore 101 by the potential gradient generated in the vicinity of the nanopore 101, as illustrated in (a) of FIG. 3A.

After the biomolecule 109 is introduced into the nanopore 101, the voltage between the electrodes 105A and 105B is switched to a second voltage V2 (Step S2 in FIG. 3B). Thus, the biomolecule 109 moves further to the downstream side, and the molecular motor 110 approaches the nanopore 101 side. Since the dimension Dm of the molecular motor 110 is larger than the diameter Dn of the nanopore 101 (Dm>Dn), when the molecular motor 110 reaches an inlet (liquid tank 104A side) of the nanopore 101, it is not possible for the molecular motor to pass through the nanopore 101 and then proceed to an outlet side (liquid tank 104B side). That is, the molecular motor 110 stops at the inlet of the nanopore 101. On the other hand, the biomolecule 109 that is charged with negative charges proceeds further in a downstream direction, and the shape of the control strand 111 changes around the spacer 113. If the shape of the control strand changes, the molecular motor 110 comes into contact with the termination of the primer 112 in the control strand 111, and then is bound to the termination of the primer 112 ((b) in FIG. 3A). Thus, the molecular motor 110 bound to the termination of the primer 112 starts the synthetic reaction.

After the molecular motor 110 and the primer 112 are bound to each other (comes into contact with each other), the voltage between both the electrodes 105A and 105B is switched to a third voltage V3 (Step S3 in FIG. 3B). Thus, an operation of pulling (transporting) the biomolecule 109 up and sequencing are started. When the synthetic reaction by the molecular motor 110 is started, the biomolecule 109 is transported in a direction opposite to an electric field direction because a force F1 of pulling the biomolecule 109 up is stronger than a force F2 when the biomolecule 109 passes through the nanopore 101. During this transport, the ammeter 106 and the computer 108 may detect (perform sequencing) a signal (change in current) derived from the characteristics of the biomolecule 109.

Regarding the applied voltage, the first voltage V1 used when the biomolecule 109 is introduced, the second voltage V2 applied when the molecular motor 110 is bound to the primer 112, and the third voltage V3 in the measurement may all be equal to each other. On the other hand, since the binding force and the pulling force differ depending on the type of molecular motor 110, using different voltages enables detection of a desired signal. The force F2 that contributes to the passing speed of the biomolecule 109 is determined by the pulling force F1, the electric field, and the frictional force on the inner wall of the nanopore 101. Therefore, it is necessary to adjust the applied voltages V1 to V3 in accordance with the dimensions (diameter, thickness, and the like) of the nanopore 101.

In the first embodiment, as an example, a polymerase is used as the molecular motor 110, and the DNA of a sequence shown in Table 1 is used as the biomolecule 109 and the primer 112. iSpC3 can be disposed as the spacer 113 at a position indicated by “Z”.

TABLE 1

| NAME | SEQUENCE |
|---|---|
| PRIMER 112 | AGCAATATCAGCACCAACAGAACACCGC |
| BIOMOLECULE | 5′- |
| 109 | ACACACACACACACACACACACACACACACACACAC |
| | ACACACACACACACZZZZACACACACACACACACAC |
| | ACACACACACACACACACACACACACACACACZZZZ |
| | GCGGTGTTCTGTTGGTGCTGATATTGCT-3′ |

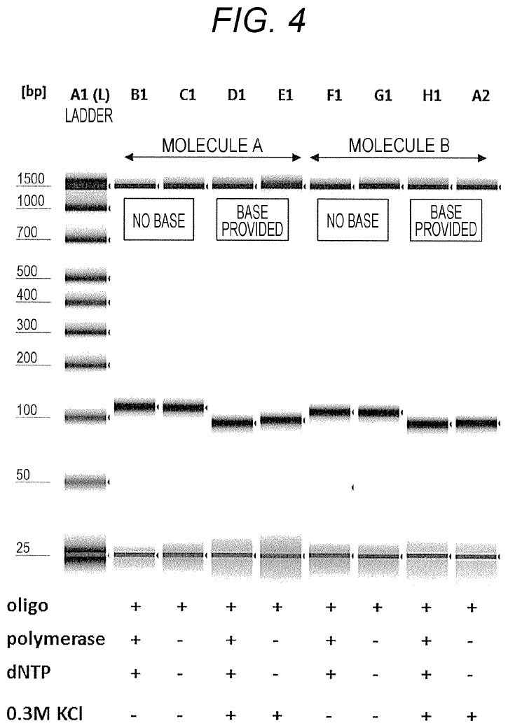

FIG. 4 illustrates an experimental example regarding the effect of the spacer 113.

In this experiment, a biomolecule 109 to which the control strand 111 was bound and which had a spacer 113 was introduced into a buffer solution. In addition, an in-solution synthesis reaction of the control strand-bound biomolecule was confirmed by electrophoresis. Further, a polymerase as a molecule used as the molecular motor 110 and dNTP for forming a complementary strand were also introduced into the buffer solution. In addition, as a comparison target (reference), the extension reaction when the polymerase used as the molecular motor 110 and the dNTP for forming the complementary strand were not introduced was also measured. As the molecule (polymerase) used as the molecular motor 110, two types of molecules, molecule A and molecule B, were examined.

In FIG. 4, “oligo +” indicates a case where the spacer 113 is provided. Further, “polymerase +” indicates an experiment in a state (+) where the polymerase as the molecular motor 110 is provided in the buffer solution. “polymerase −” indicates an experiment in a state (−) where the polymerase as the molecular motor 110 is not provided in the buffer solution. “dNTP +” indicates an experiment in a state (+) where dNTP forming the complementary strand is provided in the buffer solution. “dNTP−” indicates an experiment in a state (−) where dNTP forming the complementary strand is not provided in the buffer solution.

In addition, in order to confirm the effect of the base, the result of the extension reaction in the buffer solution in which 0.3M KCl was added to the buffer was also confirmed. In FIG. 4, “0.3M KCl +” and “0.3M KCl −” indicate whether or not 0.3M KCl is added to the buffer solution. B1 to E1 indicate the experimental results when the molecule A is used, and F1 to A2 indicate the experimental results when the molecule B is used.

For both molecules A and B, the position of a band appearing under a buffer condition (B1, F1) enabling the reaction was the same as the position of a band appearing under a buffer condition (C1, G1) where the extension reaction was not possible. From this, it was understood that the extension reaction did not occur under the buffer condition enabling the extension reaction. From the results of D1 and H1, it was understood that the extension reaction in 0.3 M KCl was also suppressed in both molecules. From the above description, it was understood that the extension reaction in the buffer solution was suppressed by providing the spacer 113 in the control strand 111 connected to the biomolecule 109.

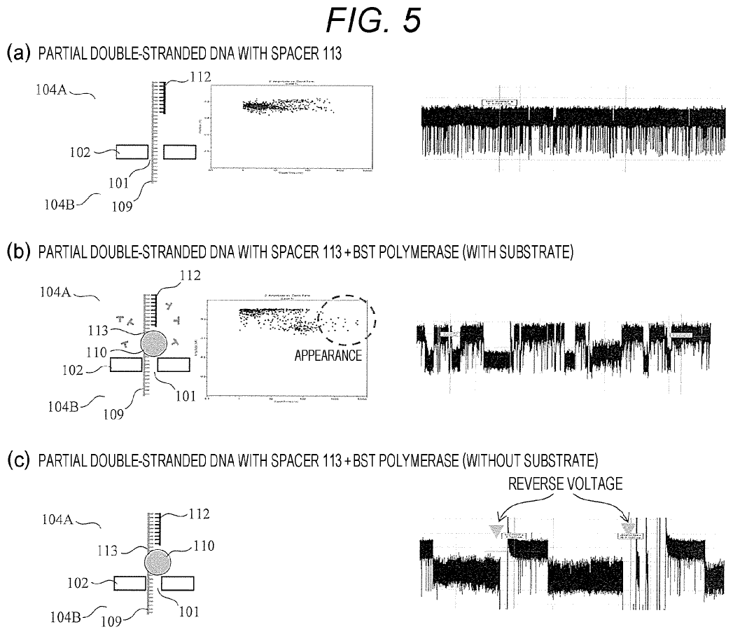

FIG. 5 illustrates data on an experiment for confirming whether or not, in the biomolecule 109 which is a partial double-stranded DNA having the molecular motor 110 and the spacer 113, the extension reaction is started when the molecular motor 110 comes into contact with the partial double-stranded DNA, and the transport of the biomolecule 109 is possible. In this experiment, the same solution conditions as those in FIG. 4 were set for the buffer solutions in the liquid tanks 104A and 104B separated by the thin film 102 having the nanopore 101. Then, the biomolecule 109 connected to the control strand 111 having the molecular motor 110 (BST polymerase) and the spacer 113, which was the same as that in FIG. 4, was dissolved in the buffer solution, and the blockade current was measured.

In the blockade current measurement, the current value measured in a state where the biomolecule 109 is not provided is used as a reference (pore current). The decrease in current, which is observed when the biomolecule 109 is encapsulated (blockade of the nanopore 101 by the biomolecule 109), is monitored, and the passing speed and the state of molecules are observed.

When the biomolecule 109 finishes passing through the nanopore 101, the acquired current value returns to the pore current being the reference. The passing speed of the nanopore 101 of the biomolecule 109 can be calculated from this blockade time, and the characteristics of the biomolecule can be analyzed from the amount of the blockade. The applied voltage used in the blockade current measurement was all set to 0.1 V in this experiment.

In the experiment using the molecular motor 110, partial double-stranded DNA was generated by previously binding the biomolecule 109 used as a template and the primer 112, and then, incubation in a buffer solution at 37° C. was performed for 10 minutes in order to perform binding to the molecular motor 110. Then, the partial double-stranded DNA bound to the molecular motor 110 was mixed with 0.3M KCl being a measurement solution. The amount of DNA was adjusted to 10 nM.

FIG. 5(a) illustrates a blockade signal and a scatter diagram illustrating a blockade time and the amount of blockade, when the biomolecule 109 which is not bound to the molecular motor 110, but bounded to only the prime 112 is measured. It can be understood that, although the waveform of the blockade signal varies, the blockade time of the acquired signal is 1 ms or less, and the biomolecule 109 passes through the nanopore 101 at a very high speed.

Next, FIG. 5(b) illustrates the result of similarly acquiring the blockade signal in a state where the biomolecule 109 is bound to the molecular motor 110 composed of the polymerase. A long-term (˜ several thousand ms) blockade signal, which was not confirmed under the conditions in FIG. 5(a), was acquired. From the scatter diagram of the blockade time and the amount of blockade, it can be understood that a plurality of signals with a blockade time of 1 ms or more are detected (indicated by a circle of a broken line). It is considered that the signal having the blockade time of 1 ms or more is based on the progress of the extension reaction after the molecular motor 110 reaches the nanopore 101.

Furthermore, FIG. 5(c) illustrates the result obtained by acquiring the blockade signal when the molecular motor 110 composed of the polymerase is bound to the biomolecule 109, but the substrate is not put in the measurement solution. A phenomenon that the decrease in current was maintained for a long time was confirmed.

After the decrease in current, that is, the blockage of the pore, was confirmed, the current value did not spontaneously return to the pore current. It was confirmed that, when the applied voltage was inverted at the time indicated by the triangle in FIG. 5(c), the current returned to the pore current. However, after a few seconds, an aspect that the current decreased again was confirmed, and the above-described phenomenon was repeated again. This phenomenon was not confirmed in FIG. 5(b).

From the above description, the following is inferred. In FIG. 5(a), as illustrated on the left side of FIG. 5A, it is inferred that the passing phenomenon of the sample in which the biomolecule 109 as a template and the primer 112 are bound is observed, and in the nanopore 101, unzipping of the primer 112 from the template is observed.

In addition, in FIG. 5(c), it is inferred that, from the result of confirming the in-solution synthesis reaction by electrophoresis in FIG. 4 described above, a state where the extension reaction by the molecular motor 110 composed of polymerase does not proceed and stops in the vicinity of nanopore 101 is observed.

On the other hand, in FIG. 5(b), it is inferred that, since the substrate is in the solution, the molecular motor 110 composed of polymerase can carry out the extension reaction from the primer 112, so that it is possible to confirm the blockade phenomenon derived from the transport by the polymerase. Note that, when the blockade time of the blockade phenomenon, which has not been confirmed in FIG. 5(b), is analyzed, the average is 1600 ms, and this means that the biomolecule passes at a speed of 30 ms/nt when converted from the length of the template of 53 nt used at this experiment. This is approximately equal to the transport time by polymerase.

From the above description, it is considered that it is possible to realize the transport stop in the solution by the spacer 113, and the transport start and the synthesis start of the biomolecule 109 in the vicinity of the nanopore 101.

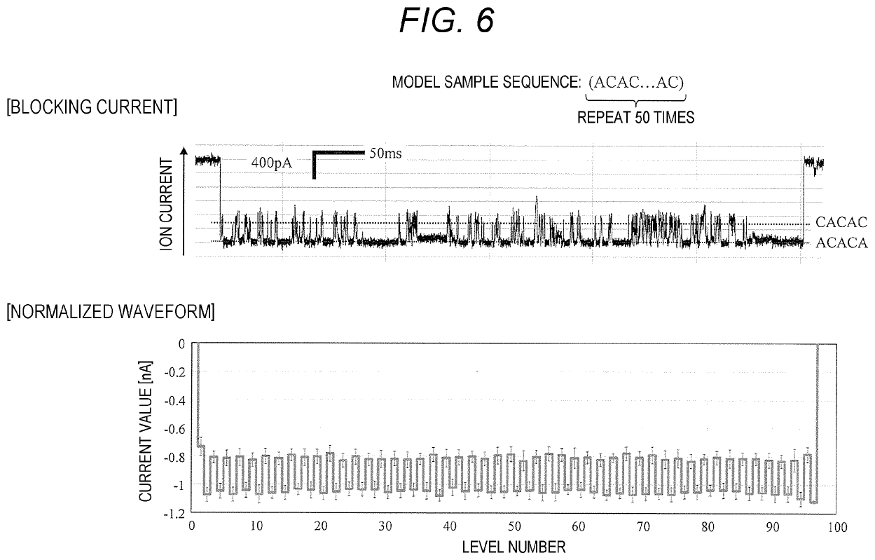

FIG. 6 illustrates a result obtained by measuring a blockade current using a biomolecule (model sample sequences) in which only adenine (A) and cytosine (C) are alternately and repeatedly formed by one base at a time (for example, 50 each), as a template biomolecule, in a similar manner to that in FIG. 5. The upper graph of FIG. 6 illustrates the waveform of the blockade current. The lower graph illustrates a normalized waveform obtained by normalizing the waveform of the blockade current in accordance with two threshold currents.

As illustrated in the upper graph of FIG. 6, when the blockade current waveform was normalized in accordance with the two threshold currents (CACAC, ACACA), and the number of pulses of the normalized waveform is counted, pulses of which the number was equal to the number of adenine and cytosine in a model sample array to be measured were observed. From the experimental results of FIG. 6, it is considered that the measured blockade current signal is a signal that reflects the characteristics (base sequence) of the template (biomolecule 109) on the downstream of the spacer 113, and the extension reaction is caused at a rate at which the difference between adjacent signals to which the base is given can be eliminated.

Then, the material of the spacer 113 will be examined. As the material of the spacer 113 contained in the control strand 111, the following materials may be used. The spacer 113 is a linear conjugate that does not contain a base. The arrangement length of the spacer 113 is required to have a length of two bases or more, that is, about 0.6×2 nm or more from a connecting portion of the primer 112. Examples of suitable linkers are well known in the field (from the IDT home page (http://sg.idtdna.com/site/Catalog/Modifications/Category/6), Diehl et al. Nature Methods, 2006, 3(7): See 551-559), and the embodiment is not limited to the examples. The embodiment is not limited to including C3 Spcer, PC spacer, Spacer9, Spacer18, dSpacer, which can be disposed in a strand. In addition to the above description, linear carbon strands, linear amino acids, linear fatty acids, linear sugar strands and the like may be used.

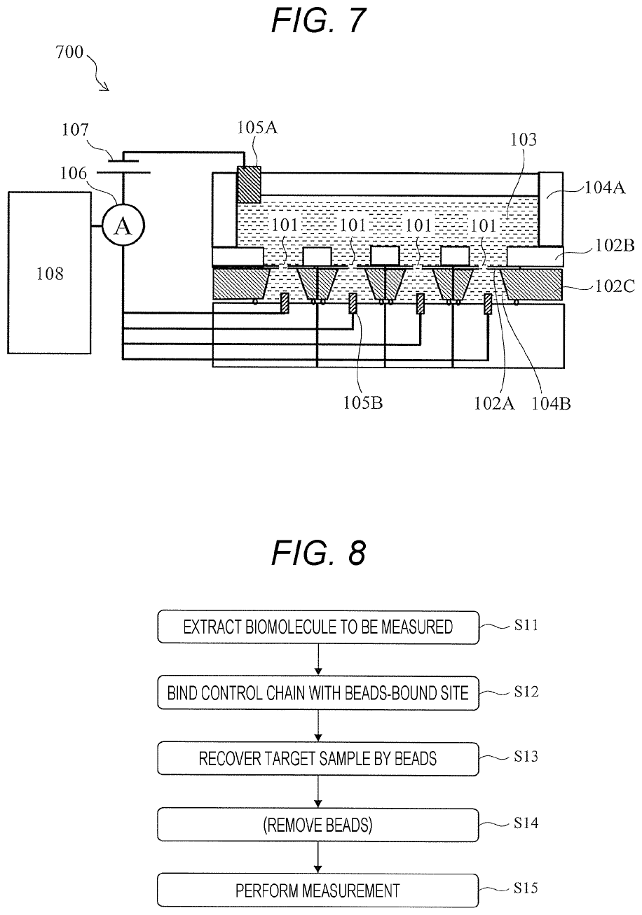

FIG. 7 illustrates an example of a configuration of a biomolecule analysis device 700, which is different from the biomolecule analysis device in FIG. 1. Since the same components as those in FIG. 1 are denoted by the same reference signs, repetitive description will be omitted. The difference from the device in FIG. 1 is that the thin film 102B has a plurality of nanopores 101, and the liquid tank 104B under the thin film 102A is divided into a plurality of spaces by partition walls (specifically, side wall of the thin film 102C). In thin films 102B and 102C for fixing the thin film 102A, through-holes are provided at positions corresponding to the nanopores 101, and the plurality of spaces are formed by the side walls of the through-holes of the thin film 102C. The second electrode 105B is provided in each of the plurality of spaces. Note that, the liquid tank 104A is used as a common liquid tank for the plurality of spaces located on the lower side. The plurality of spaces are insulated from each other by partition walls. Therefore, it is possible to independently measure the current flowing through each nanopore 101.

The thin film 102A exposed in each of the through-holes provided in the thin films 102B and 102C preferably has an area in which it is difficult to form two or more nanopores 101 when forming the nanopore 101 by applying a voltage. This area is allowed in terms of strength. As an example, the area can be, for example, about 100 to 500 nm. Further, the film thickness of the thin film 102A is preferably set to a film thickness enabling forming of the nanopore 101 having an effective film thickness equivalent to one base in order to achieve a single base resolution of DNA. As an example, it is appropriate that the film thickness is set to about 7 nm or less.

The liquid tank 104A and the liquid tank 104B are filled with the electrolyte solution 103, similar to the case in FIG. 1. In the case of FIG. 7, the volume of the electrolyte solution 103 is on the order of microliters or milliliters.

For example, KCl, NaCl, LiCl, and CsCl are used for the electrolyte solution 103. Regarding the solution, urea of 4M or more, DMSO, DMF, and NaOH can be mixed in the liquid tank 104B into which the molecular motor 110 is not introduced, in order to suppress the formation of self-complementary strands of the biomolecule 109. Further, it is possible to mix a buffer to stabilize the biomolecule 109. As the buffer, Tris, EDTA, PBS, and the like are used.

A method of manufacturing the biomolecule analysis device described above will be described below. The basic configuration itself of the biomolecule analysis device used for analyzing the biomolecule with the so-called blockade current method is known in the art, and the components thereof can be easily understood by those skilled in the art. For example, U.S. Pat. No. 5,795,782, “Scientific Reports 4, 5000, 2014, Akahori, et al.”, “Nanotechnology 25 (27): 275501, 2014, Yanagi, et al.”, “Scientific Reports, 5, 14656, 2015, Goto, et al.”, and “Scientific Reports 5, 16640, 2015” disclose the specific devices.

The thin film 102 on which the nanopore 101 is formed may be a lipid bilayer (biopore) composed of an amphipathic molecular layer in which a protein having a pore in the center is embedded, or may be a thin film (solid pore) formed of a material that can be formed by a semiconductor microfabrication technology. Examples of the material that can be formed by the semiconductor microfabrication technology include silicon nitride (SiN), silicon oxide (SiO₂), silicon nitride (SiON), hafnium oxide (HfO₂), molybdenum disulfide (MoS₂), and graphene. The thickness of the thin film is 1 Å (angstrom) to 200 nm, preferably 1 Å to 100 nm, more preferably 1 Å to 50 nm, and, for example, about 5 nm.

As an example, a thin film produced by the semiconductor microfabrication technology can be produced by the following procedure. Firstly, Si₃N₄/SiO₂/Si₃N₄ are formed on the front surface of an 8-inch Si wafer having a thickness of 725 μm in order with film thicknesses of 12 nm/250 nm/100 nm, respectively. In addition, Si₃N₄ is deposited at 112 nm on the back surface of the Si wafer.

Then, Si₃N₄ at the top of the front surface of the Si wafer is removed by reactive ion etching at 500 nm square. Similarly, Si₃N₄ on the back surface of the Si wafer is removed by reactive ion etching at 1038 μm square. On the back surface, the Si substrate exposed by etching is further etched with TMAH (Tetramethylammonium hydroxide). During Si etching, preferably, the wafer surface is covered with a protective film (ProTEKTMB3 primer and ProTEKTMB3, Brewer Science, Inc.) in order to prevent etching of SiO on the front surface side. SiO of the intermediate layer may be polysilicon.

Then, after the protective film is removed, the SiO layer exposed at 500 nm square is removed with a BHF solution (HF/NH₄F=1/60, 8 min). Thus, a partition body in which the thin film Si₃N₄ having a film thickness of 12 nm is exposed is obtained. When polysilicon is selected for a sacrificial layer, the thin film is exposed by etching with KOH. At this stage, the nanopore is not provided on the thin film.

Regarding the dimensions of Nanopore 101, an appropriate dimension can be selected in accordance with the type of biomolecule to be analyzed. As an example, the dimensions of the nanopore 101 can be set to, for example, 0.9 nm to 100 nm, preferably 0.9 nm to 50 nm, and specifically, about 0.9 nm or more and 10 nm or less. For example, the diameter of the nanopore 101 used for the analysis of ssDNA (single-stranded DNA) having a diameter of about 1.4 nm can be set to, preferably about 1.4 nm to 10 nm, more preferably about 1.4 nm to 2.5 nm, and specifically, about 1.6 nm.

In addition, for example, the diameter of the nanopore 101 used for the analysis of dsDNA (double-stranded DNA) having a diameter of about 2.6 nm can be set to, preferably about 3 nm to 10 nm, and more preferably about 3 nm to 5 nm.

The depth of the nanopore 101 can be adjusted by adjusting the thickness of the thin film. The depth of the nanopore 101 is set to be at least twice the monomer unit constituting the biomolecule, preferably at least three times, more preferably at least five times. For example, when the biomolecule is composed of nucleic acid, the depth of nanopore 101 is set to be preferably a size of 3 or more bases, for example, about 1 nm or more. Thus, it is possible to enter biomolecules into the nanopore 101 while controlling the shape and the moving speed of the biomolecule, thereby highly sensitive and accurate analysis is possible. In addition, the shape of the nanopore 101 is basically circular, but can also be elliptical or polygonal.

In the case of an array-type device configuration including a plurality of thin films having nanopores 101, it is preferable that the thin films having nanopores 101 be regularly arranged. The interval at which the plurality of thin films 111A are arranged can be set to 0.1 μm to 10 μm, and preferably 0.5 μm to 4 μm, depending on the electrodes to be used and the capabilities of the electrical measurement system.

Note that, the method for forming the nanopore 101 in the thin film is not particularly limited. For example, electron beam irradiation by a transmission electron microscope or dielectric breakdown by voltage application can be used. For example, the method disclosed in “Itaru Yanagi et al., Sci. Rep. 4, 5000 (2014)” can be used.

The nanopore 101 can be formed, for example, by the following procedure. Before setting the partition body on the device for biomolecule analysis, a Si₃N₄ thin film is hydrophilized under the conditions of 10 W, 20 sccm, 20 Pa, and 45 sec by Ar/O₂ plasma (SAMCO Inc., Japan). Then, the partition body is set in the device for biomolecular analysis. Then, the upper and lower liquid tanks sandwiching the thin film are filled with a solution of 1M KCl, 1 mM Tris-10 mM EDTA, and pH 7.5, and the electrodes 115A and 115B are introduced into the respective liquid tanks.

The voltage is applied not only when the nanopore 101 is formed, but also when the ionic current flowing through the nanopore 101 is measured after the nanopore 101 is formed. Here, the liquid tank located on the lower side is referred to as a cis tank, and the liquid tank located on the upper side is referred to as a trans tank. In addition, a voltage Vc is applied to the electrode on the cis tank side is set to 0 V, and a voltage Vtrans is applied to the electrode on the trans tank side. The voltage Vtrans is generated by a pulse generator (for example, 41501B SMU AND Pulse Generator Expander, Agilent Technologies, Inc.).

The current value after pulse application can be read with an ammeter (for example, 4156B PRECISION SEMICONDUCTOR ANALYZER, Agilent Technologies, Inc.). The current value condition (threshold current) can be selected in accordance with the diameter of the nanopore 101 formed before the application of the pulse voltage, and the desired diameter can be obtained while sequentially increasing the diameter of the nanopore 101.

The diameter of the nanopore 101 is estimated from the ionic current value. The criteria for selecting conditions are shown in Table 2.

TABLE 2
VOLTAGE APPLICATION CONDITION

| | DIAMETER OF NANOPORE BEFORE | | |
|---|---|---|---|
| | APPLICATION OF PULSE VOLTAGE | | |
| | NOT-OPEN~0.7 nmΦ | ~1.4 nmΦ | ~1.5 nmΦ |
| APPLIED VOLTAGE | 10 | 5 | 3 |
| (V_{cis}) [V] | | | |
| INITIAL | 0.001 | 0.01 | 0.001 |
| APPLICATION | | | |
| TIME [s] | | | |
| THRESHOLD | 0.1 nA/ | 0.6 nA/ | 0.75 nA/ |
| CURRENT | 0.4 V | 0.1 V | 0.1 V |

Here, the n-th pulse voltage application time to (where n>2 is an integer) is determined by the following expression.

tₙ=10^{-3+(1/6)(n-1)}−10^{-3+(1/6)(n-2)} For n>2 [Math. 1]

The nanopore 101 can be formed by electron beam irradiation with TEM (A. J. Storm et al., Nat. Mat. 2 (2003)) in addition to the method of applying a pulse voltage.

When a voltage is applied from the power source to the electrodes provided in the upper and lower liquid tanks, an electric field is generated in the vicinity of the nanopore 101, and the biomolecule that is negatively charged in the liquid passes through the nanopore 101. At this time, the blockade current Ib described above flows.

The liquid tank that can store the measurement solution that comes into contact with the thin film can be appropriately provided with a material, a shape, and a size that do not affect the measurement of the blockade current. The measurement solution is injected to come into contact with the thin film that partitions the liquid tanks.

The electrode is preferably produced with a material capable of causing an electron transfer reaction (Faraday reaction) with the electrolyte in the measurement solution, and is typically produced with silver halide or silver halide. From the viewpoint of potential stability and reliability, it is preferable to use silver or silver-silver chloride.

The electrode may be produced with a material that serves as a polarization electrode, and may be produced with, for example, gold or platinum. In this case, preferably, a substance capable of assisting the electron transfer reaction, for example, potassium ferricyanide or potassium ferrocyanide, is added to the measurement solution in order to secure a stable ionic current. Alternatively, it is preferable to immobilize a substance capable of carrying out the electron transfer reaction, for example, ferrocenes, on the surface of the polarization electrode.

The structure of the electrode may be entirely made of the above-described material, or the surface of a base material (copper, aluminum, etc.) may be coated with the above-described material. The shape of the electrode is not particularly limited, but a shape having a large surface area in contact with the measurement solution is preferable. The electrodes are joined to the wiring, and an electrical signal is transmitted to a measurement circuit.

The biomolecule analysis device in FIG. 7 includes the above components as elements. The above-described nanopore-type biomolecule analysis device may be provided together with a manual describing the procedure and amount of use. The control strand may be provided in a ready-to-use state, or may be configured and provided in a state in which only the biomolecule to be measured is not bound. Such forms and preparations can be understood by those skilled in the art. Similarly, a nanopore device may be provided in a state where the nanopore is formed in a ready-to-use state, or may be provided in a state where the nanopore is formed at the providing destination.

FIG. 8 is a flowchart illustrating a measurement procedure by the biomolecule measuring devices 100 and 700. Firstly, the biomolecule 109 to be measured is extracted from the solution (Step S11), and the biomolecule 109 is bound to the control strand 111 (Step S12). The control strand 111 can be modified with a molecule capable of binding to a modifying group on the surface of the bead, which will be used later for recovery. The biomolecule 109 can be obtained, for example, by being extracted from a cell fluid of the target organism. After the extraction, the biomolecule 109 is bound to the control strand 111 and is recovered. It is common to use beads in recovery, and the surface of the beads is modified with a molecule capable of being bound to a molecule modified to a control strand.

For example, streptavidin is often bound to the bead surface. In this case, since the 3′end of the control strand 111 is modified with biotin, only the sample to which the control strand 111 can be connected can be recovered with beads. The binding target of streptavidin and biotin may be reversed. After collecting the target sample (biomolecule 109 to be measured) with the beads (Step S13), the beads may be removed in some cases (Step S14). When removing the beads, it is also possible to remove the beads by applying an electric field of 800 mV or more to both ends of a membrane having a porous structure smaller than the bead diameter. Alternatively, it is also possible to be dissociated with a reducing agent by preparing a disulfide bond site on the downstream of a SA-biotin bond.

Then, the extracted biomolecule 109 is dissolved in the measurement solution and the measurement solution is injected into the biomolecule analysis device illustrated in FIG. 1 or 7. Then, the biomolecule 109 is introduced into the nanopore 101 by applying a voltage, and the biomolecule 109 is analyzed by applying a desired voltage.

As described above, according to the biomolecule analysis device and the analysis method in the first embodiment, it is possible to precisely control the synthesis start point while controlling the transport of biomolecules.

### Second Embodiment

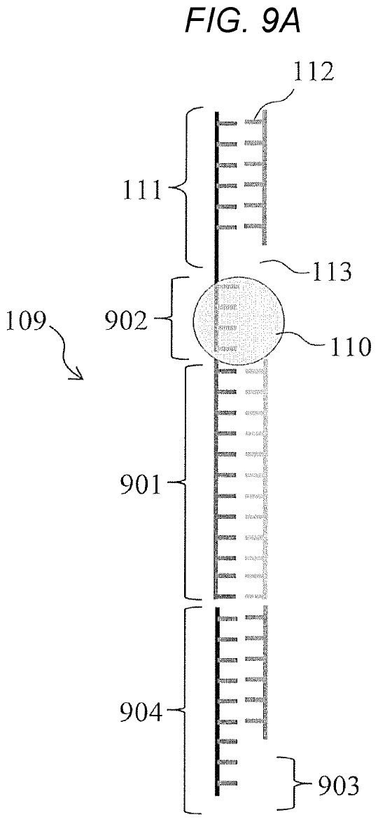

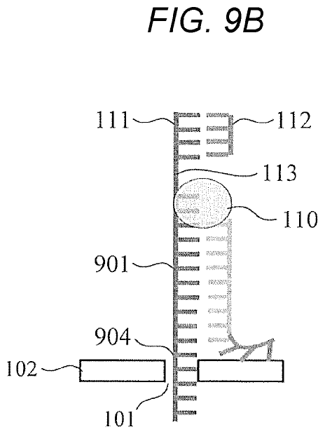

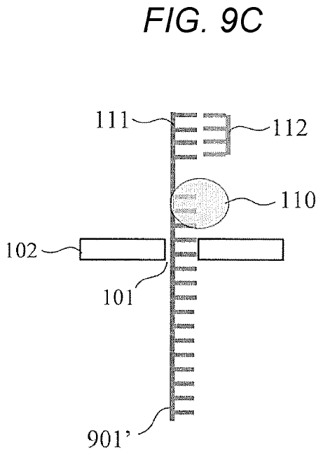

Next, a biomolecule analysis device according to a second embodiment of the present invention will be described with reference to FIGS. 9A to 9C. Since the overall configuration of the biomolecule analysis device in the second embodiment is similar to that in the first embodiment, repetitive description will be omitted. In the second embodiment, the composition of the biomolecule 109 to be measured is different from that in the first embodiment.

In the first embodiment, a case where the biomolecule 109 of single-stranded DNA is to be measured has been described as an example. In the second embodiment, a biomolecule 109 of double-stranded DNA is to be measured.

In the case of double-stranded DNA, it is not possible for the biomolecule 109 to be introduced into the nanopore 101 by electrophoresis without being treated. Therefore, in the second embodiment, as illustrated in FIG. 9A, an introductory strand 904 is added to one end of a genome fragment 901 of the double-stranded as the measurement target. The introductory strand 904 has at least a double-stranded structure on the side of the genome fragment 901 to be measured. However, the tip portion of the introductory strand 904 on the opposite side of the genome fragment 901 is a protruding termination 903 of the single strand so that introduction into the nanopore 101 is possible.

The control strand 111 is connected to the proximal end side of the genome fragment 901 through a molecular motor binding site 902 including the molecular motor 110, similar to the first embodiment. The primer 112 is added to the control strand 111 as in the first embodiment, and the spacer 113 is provided between the primer 112 and the molecular motor 110, similar to the first embodiment.

Next, the outline of the method for measuring the biomolecule 109, which has been described with reference to FIG. 9A in the biomolecule analysis device in the second embodiment will be described with reference to FIGS. 9B and 9C.

When the genome fragment 901 in FIG. 9A is introduced into, for example, the liquid tank 104A of the device in FIG. 1, and a voltage is applied between both the electrodes 105A and 105B, the introductory strand 904 of the genome fragment 901 in FIG. 9A is introduced into the nanopore 101.

As illustrated in FIG. 9B, when the protruding termination 903 of the introductory strand 904 is introduced into the nanopore 101, the double-stranded DNA of the introductory strand 904 is unzipped, and then the double-stranded DNA of the genome fragment 901 is also unzipped. Then, as illustrated in FIG. 9C, when the molecular motor 110 reaches the nanopore 101, the molecular motor 110 and the primer 112 come into contact with each other, and thereby the extension reaction is started in a single-stranded genome fragment 901′. The subsequent operations are substantially the same as those in the first embodiment. According to the configuration of the second embodiment, it is also possible to analyze a biomolecule having a double-stranded structure, by the nanopore type device as illustrated in FIG. 1.

### Third Embodiment

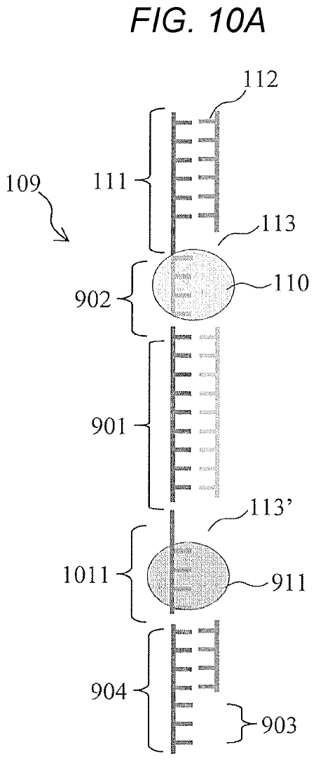

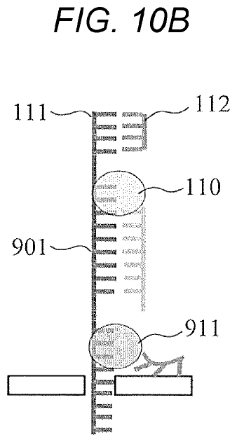

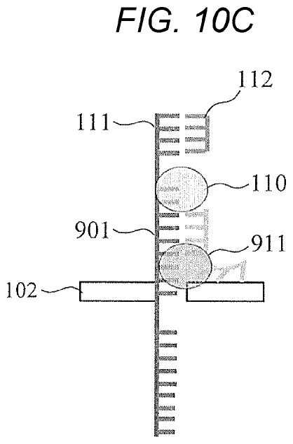

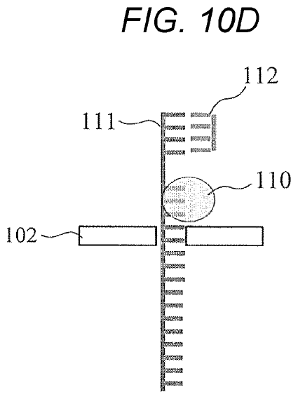

Next, a biomolecule analysis device according to a third embodiment of the present invention will be described with reference to FIGS. 10A to 10D. Since the overall configuration of the biomolecule analysis device in the third embodiment is similar to that in the first embodiment, repetitive description will be omitted. In the third embodiment, the composition of the biomolecule to be measured is different from that in the first embodiment. The same configurations as those of the biomolecule in the second embodiment are denoted by the same reference signs in FIG. 10A, and repetitive description will be omitted below.

In a biomolecule 109 in the third embodiment, the introductory strand 904 is added to one end of the genome fragment 901 to be measured, similar to the first embodiment. However, in the third embodiment, a second control strand 1011 is connected between the introductory strand 904 and the genome fragment. The second control strand 1011 includes a second molecular motor 911, and a spacer 113′ is provided at a connecting portion with the genome fragment 901. That is, in the third embodiment, the genome fragment 901 as a biomolecule is connected to the control strand 111 and the first molecular motor 110 at a first end portion, and the first molecule motor 110 and the second molecule motor 911 are connected to each other at a second end portion. The second molecular motor 911 has a function of dissociating the double strand or decomposing the complementary strand. The first molecular motor 110 is a polymerase, and the second molecular motor 911 is a helicase.

Next, in the third embodiment, a method of introducing the biomolecule 109 into the nanopore 101 will be described. Similar to FIG. 9A, the control strand 111 is bound to the upstream of the biomolecule 109 to be measured through the molecular motor binding site 902. The second control strand 1011 including the molecular motor 911 that carries out double strand dissociation or decomposes the complementary strand of a biomolecule and the spacer 113′ provided on the upstream of the molecular motor 911 is bound to the downstream of the biomolecule 109. The introductory strand 904 is connected to the downstream of the second control strand 1011.

The biomolecule 901 is extracted from a living cell to be inspected. The first control strand 111, the molecular motor binding site 902, and the second control strand 1011 to which the introductory strand 904 is connected are bound to the biomolecule 901, and then the biomolecule 109 is recovered. Then, the recovered biomolecule 109 is introduced into the liquid tank 104A in contact with the thin film 102. When a voltage is applied to the thin film 102, the biomolecule 109 is introduced into the nanopore 101 from the introductory strand 904. Thus, as illustrated in FIG. 10B, dissociation of the double-stranded structure of the introductory strand 904 is started.

Then, when the dissociation of the introductory strand 904 is completed, and the second molecular motor 911 reaches the nanopore 101, the second molecular motor 911 composed of a helicase comes into contact with the complementary strand of the genome fragment 901. Thus, as illustrated in FIG. 10C, dissociation and decomposition of the complementary strand of the genome fragment 901 are started. When the dissociation or the decomposition of the complementary strand of the genome fragment 901 is ended, the molecular motor 911 is released from the genome fragment 901.

When the decomposition or the dissociation of the genome fragment 901 is complete, the first molecular motor 110 composed of a polymerase reaches the nanopore 101, as illustrated in FIG. 10D. Thus, similar to the first embodiment, the extension reaction may be started by the contact between the primer 112 and the first molecular motor 110, and the analysis of the single-stranded genome fragment 901 may be started.

As described above, according to the third embodiment, it is possible to start the dissociation of the genome fragment 901 which is double-stranded DNA by the second molecular motor 911, after the dissociation of the introduced strand 904 is ended. Thus, it is possible to rapidly and accurately analyze double-stranded DNA in the nanopore method.

### Fourth Embodiment

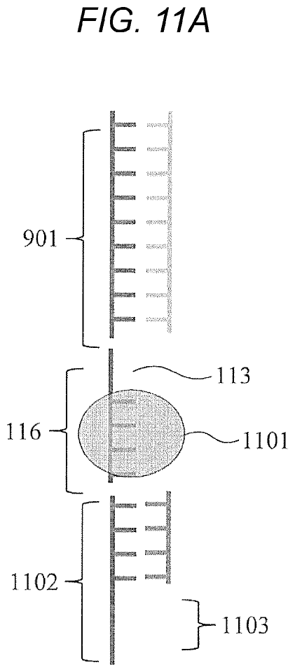

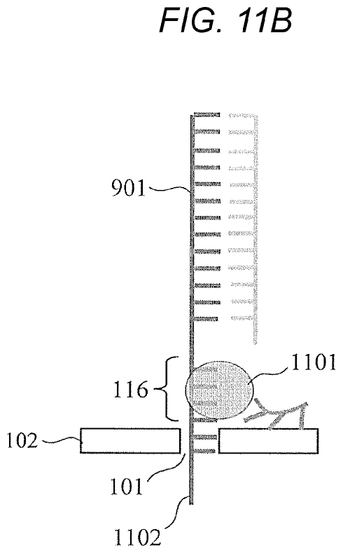

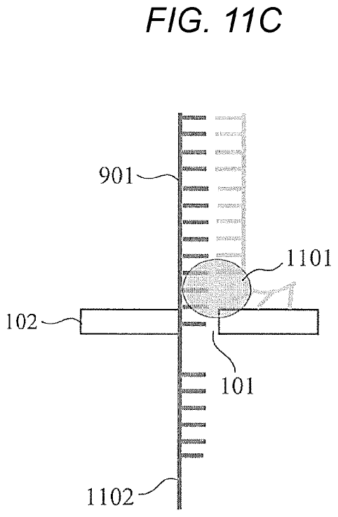

Next, a biomolecule analysis device according to a fourth embodiment of the present invention will be described with reference to FIG. 11A. Since the overall configuration of the biomolecule analysis device in the fourth embodiment is similar to that in the first embodiment, repetitive description will be omitted. In the fourth embodiment, the composition of the biomolecule to be measured is different from that in the first embodiment. In FIG. 11A, the same configurations as those of the biomolecule in the fourth embodiment are denoted by the same reference signs in FIG. 1, and repetitive description will be omitted below.

In the fourth embodiment, a molecular motor binding site 116 is connected to one end of the double-stranded genome fragment 901 to be measured, and an introductory strand 1102 is connected to the other end of the molecular motor binding site 116. The molecular motor binding site 116 contains a molecular motor 1101 composed of a helicase. This point is different from a point that the molecular motor composed of a polymerase is provided in the above-described embodiments. Differing from a polymerase, a molecular motor composed of a helicase can dissociate a double-stranded biomolecule to obtain a single-stranded structure. The molecular motor 1101 composed of a helicase is bound to a single-strand region of the control strand in the molecular motor binding site 116. The introductory strand 1102 has a protruding termination 1103. A spacer is used as the protruding termination 1103 instead of a single strand.

Next, in the fourth embodiment, a method of introducing the genome fragment 901 into the nanopore 101 will be described with reference to FIGS. 11B and 11C. Firstly, the genome fragment 901 is extracted from a cell having the genome to be measured and is purified. Then, the molecular motor binding site 116 to which the introductory strand 1102 is bound is bound. Note that, a site capable of binding to a mechanism, such as beads, for extracting a biomolecule may be bound to one end of the genome fragment 901, on an opposite side of a side to which the molecular motor binding site 116 is bound.

Then, after the mechanism for extracting the genome fragment 901 is cut, the extracted genome fragment 901 is introduced into a measurement solution in contact with the thin film 102 having the nanopore 101. In the device in FIG. 1 or FIG. 7, when a voltage is applied between the electrodes 105A and 105B, the introductory strand 1102 of the genome fragment 901 is introduced into the nanopore 101. When the introductory strand 1102 is introduced into the nanopore 101, as illustrated in FIG. 11B, the complementary strand of the double-stranded region starts dissociation. When the dissociation of the complementary strand is ended, and the molecular motor 1101 composed of a helicase reaches the nanopore 101, the molecular motor 1101 comes into contact with one end of the genome fragment 901. Thus, the dissociation of the complementary strand by the helicase is started, and the genome fragment 901 becomes a single strand and can pass through the nanopore 101. Accordingly, the genome fragment 901 is transported inside the nanopore 101, and the analysis of the genome fragment 901 by the nanopore method is started.

### Fifth Embodiment

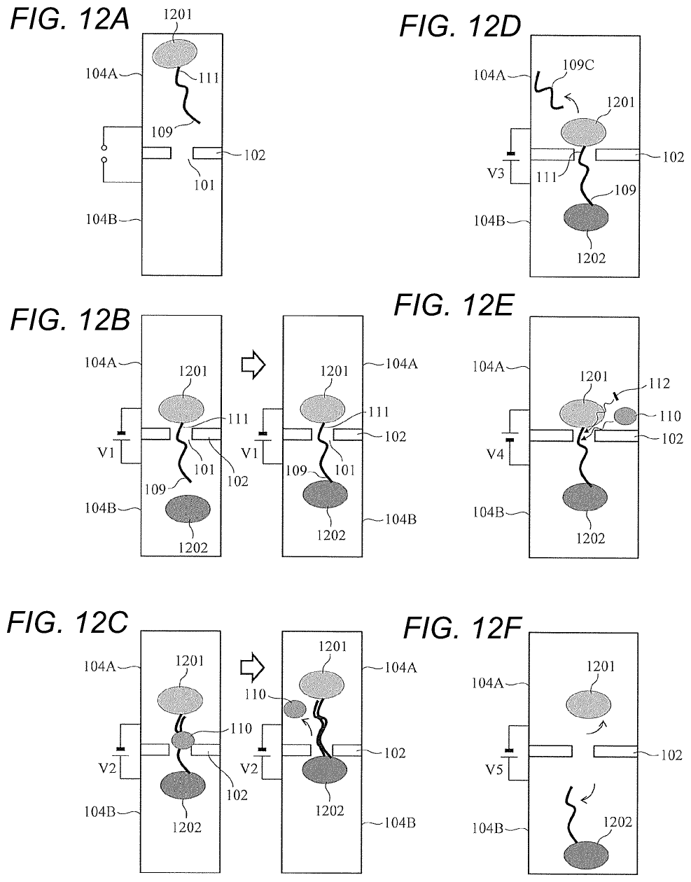

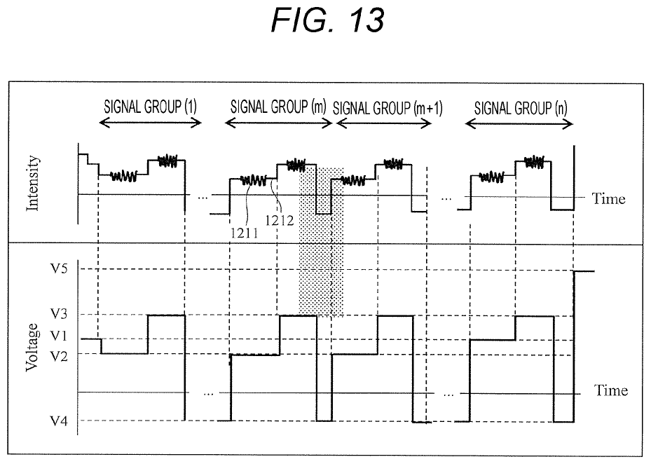

Next, a biomolecule analysis device according to a fifth embodiment of the present invention will be described with reference to FIGS. 12 and 13. Since the overall configuration of the biomolecule analysis device in the fifth embodiment is similar to that in the first embodiment, repetitive description will be omitted.

In the fifth embodiment, the composition of the biomolecule to be measured and the operation of the device are different from those in the first embodiment. In the fifth embodiment, in order to improve the analysis accuracy of the biomolecule, the biomolecule is configured so that the reciprocating control capable of repeatedly analyzing the biomolecule can be executed. Here, the reciprocating control refers to a control in which the biomolecule 109 moves up and down in the nanopore 101 a plurality of times to enable repeated measurement of one biomolecule 109.

In addition, the biomolecule analysis device is configured to provide a voltage application that allows such reciprocating control. Specifically, the reciprocating control is possible by binding stopper molecules 1201 and 1202, which are larger in size than the nanopore 101, to both ends of the biomolecule 109. It is possible to repeatedly measure the same biomolecule by performing the reciprocating control. Thus, it is possible to improve the measurement accuracy.

An outline of a procedure for analyzing a biomolecule by the reciprocating control in the fifth embodiment will be described with reference to FIG. 12. Reciprocating analysis by reciprocating control is realized by trapping the biomolecule 109 in the vicinity of the nanopore 101 and repeatedly reversing the polarity of voltage application to perform reciprocating movement.

Firstly, as illustrated in FIG. 12A, a first stopper molecule 1201 is bound to one end of the biomolecule 109 to be measured through the control strand 111, and is enclosed in the first liquid tank 104A.

Then, as illustrated in FIG. 12B, when a first voltage (V1) is applied between the electrodes 105A and 105B, the biomolecule 109 is introduced (passed) into the nanopore 101 by electrophoresis. Since the size of the first stopper molecule 1201 is larger than the diameter of the nanopore 101, it is not possible for the first stopper molecule 1201 to pass through the nanopore 101, and the biomolecule 109 is trapped in the nanopore 101.

When the biomolecule 109 passes through the nanopore 101, as illustrated on the right side of FIG. 12B, a second stopper molecule 1202 enclosed in the second liquid tank 104B is bound to the other end of the biomolecule 109. In this manner, the biomolecule 109 is connected to the first stopper molecule 1201 and the second stopper molecule 1202, respectively, with both ends of the biomolecule 109 located on both sides of the thin film 102, so that the biomolecule 109 can reciprocate inside the nanopore 101 between the first stopper molecule 1201 and the second stopper molecule 1202.

When the biomolecule 109 passes through the nanopore 101 and both ends are connected by the first and second stopper molecules 1201 and 1202, a primer binding site having a molecular motor 110 is subsequently bound to the biomolecule 109. Note that, it is easy to bind the primer 112 to the molecular motor 110 in the control strand 111 by reversing the polarity of the first voltage V1.

Next, as illustrated in FIG. 12C, when a second voltage V2 is applied between the electrodes 105A and 105B instead of the first voltage V1, and the molecular motor 110 reaches the nanopore 101, the molecular motor 110 and the prime 112 in the control strand 111 come into contact with each other. Thus, the extension reaction of the biomolecule 109 is started, and the biomolecule is analyzed in the similar manner to that in the first embodiment. When the extension reaction proceeds to the termination of the biomolecule 109, the molecular motor 110 is released from the biomolecule 109 (see the right side of FIG. 12C).

Then, as illustrated in FIG. 12D, when a third voltage V3 is applied instead of the second voltage V2, the complementary strand 109C of the biomolecule 109 synthesized by the molecular motor 110 is unzipped by the action of the voltage V3, and is released from the biomolecule 109.

Subsequently, as illustrated in FIG. 12E, when a fourth voltage V4 having a polarity different from that of the third voltage V3 is applied instead of the third voltage V3, the primer 112 and the molecular motor 110 are bound to the control strand 111, and thus the biomolecule 109 returns to the state illustrated in FIG. 12B. Then, the measurement of the biomolecule 109 is repeated again by applying the second voltage V2.

After a sufficient number of measurements have been repeated for one biomolecule 109, a fifth voltage V5 is applied between the electrodes 105A and 105B instead of the fourth voltage V4, as illustrated in FIG. 12F. Thus, the first stopper molecule 1201 or the second stopper molecule 1202 is separated from the biomolecule 109. Thus, the above-described measurement can be started for a new biomolecule different from the biomolecule 109 for which the measurement has been completed. Here, the fifth voltage V5 is a voltage capable of generating an electric field having a force which is larger than the force of the binding site between the stopper molecules 1201 and 1202, and the biomolecule 109. For example, when a bond by Streptavidin and biotin is used, cutting can be performed when a voltage of 800 mV is applied in a case of using a nanopore having a film thickness of 7 nm.

FIG. 13 is a graph illustrating a change in waveform of a signal obtained when the steps in FIGS. 12(a) to 12(f) are executed, and a change in the waveform of the applied voltage. In FIG. 13, a similar voltage waveform is repeatedly input. When such a voltage waveform is repeatedly input, a plurality of signal groups can be obtained as a result, thereby it is possible to improve the measurement accuracy of the biomolecule 109 to be measured. Note that, the signal group includes a signal derived from the biomolecule and a signal for determining that the transport control has stopped.

The computer 108 detects the current value detected by the ammeter 106. When a predetermined current or a voltage corresponding to the current is detected, the value of the voltage applied between the electrodes 105A and 105B is switched with the detection as the trigger (V1→V2→V3→V4).

For example, in FIG. 13, in a signal group (m), when the second voltage V2 is applied, a signal 1211 derived from the biomolecule 109 to be analyzed is obtained. The signal 1211 is a signal that vibrates in accordance with the sequence and the like of nucleic acids of the biomolecule 109.

Then, when the biomolecule 109 moves and the analysis is ended up to the termination of the biomolecule 109, the vibration of the signal 1211 based on the base sequence is ended, and the voltage is settled at a substantially constant voltage 1212. When the computer 108 detects the predetermined voltage 1212, the computer 108 switches the voltage from the second voltage V2 to the third voltage V3 being a voltage for returning the biomolecule 109 to the initial position. Even in the process of returning the biomolecule 109 to the initial position, the current value detected by the ammeter 106 varies depending on the structure of the biomolecule 109. When this current value settles to a predetermined value, it is determined that the return to the initial position is completed. Thus, the applied voltage V3 is switched to the fourth voltage V4 having the opposite characteristics. Thus, the primer 112 and the molecular motor 110 are bound to the biomolecule 109 again. The re-measurement of the biomolecule 109 is started, and a signal group (m+1) is obtained. When the measurement of the biomolecule 109 is ended a plurality of number of times (n times), the fifth voltage V5 is applied, and thereby the measurement of one biomolecule 109 is ended.

As described above, the biomolecule 109 can be obtained, for example, by extracting the biomolecule from a cell fluid of the target organism. After the extraction, the biomolecule 109 is bound to the control strand 111 and is recovered. It is common to use beads in recovery, and the surface of the beads is modified with a molecule capable of being bound to a molecule modified to a control strand. As the stopper molecules 1201 and 1202 described above, the beads itself used for recovering the control strand binding molecule can be used. Alternatively, it is also possible to perform measurement through a process of removing the biomolecule after recovery with beads and binding the stopper molecule again.

Hitherto, some embodiments of the present invention have been described above, but the embodiments are presented as examples and are not intended to limit the scope of the invention. The novel embodiments can be implemented in various other forms, and various omissions, replacements, and changes can be made without departing from the spirit of the invention. The embodiments and their modifications are included in the scope and the gist of the invention, and are also included in the invention described in the claims and the scope of equivalents thereof.

### REFERENCE SIGNS LIST

100, 700 biomolecule analysis device
101 nanopore
102 thin film
103 electrolyte solution
104A, 104B liquid tank
105A first electrode
105B second electrode
106 ammeter
107 voltage source
108 computer
109 biomolecule (DNA strands, and like)
110, 911, 1101 molecular motor
111, 1011 control strand
112 primer
113, 113′ spacer
116, 902 molecular motor binding site
901 genome fragment
903, 1103 protruding termination
1201, 1202 stopper molecule
904, 1102 introductory strand

1. A biomolecule analysis device comprising:
a thin film having a nanopore;
a liquid tank that is disposed in contact with the thin film and contains an electrolyte solution;
an electrode in contact with the liquid tank;
a measurement device connected to the electrode; and
a controller that controls a voltage to be applied to the electrode, in accordance with a measurement result of the measurement device,
wherein a biomolecule is introduced into the electrolyte solution,
a control strand and a molecular motor are connected to a first end portion of the biomolecule, and
the control strand is bound to a primer on an upstream of the control strand and has a spacer on a downstream of the control strand.

2. The biomolecule analysis device according to claim 1, wherein a dimension of the molecular motor is larger than a size of the nanopore.

3. The biomolecule analysis device according to claim 1, wherein the biomolecule further contains an introductory strand for an introduction to the nanopore, at a second end portion of the biomolecule, and
the introductory strand has a double-stranded structure at at least an end portion on a side of the biomolecule, and has a single-stranded structure at an end portion on an opposite side of the biomolecule.

4. The biomolecule analysis device according to claim 1, wherein the first end portion of the biomolecule is connected to the control strand and the molecular motor as a first molecular motor,
a second end portion of the biomolecule, which is different from the first end portion, is connected to a second molecular motor different from the first molecular motor,
the first molecular motor is located between the spacer as a first spacer and the first end portion of the biomolecule, and
the second molecular motor is located between a second spacer and the second end portion of the biomolecule.

5. The biomolecule analysis device according to claim 4, wherein the first molecular motor is a polymerase, and
the second molecular motor is a helicase.

6. The biomolecule analysis device according to claim 1, wherein stopper molecules are further connected to both ends of the biomolecule, and
dimensions of the stopper molecules are larger than a size of the nanopore.

7. The biomolecule analysis device according to claim 1, wherein the liquid tank includes a first liquid tank located on a first surface side of the thin film and a second liquid tank located on a second surface side of the thin film,
the second liquid tank is divided into a plurality of liquid tanks by a partition wall, and
the biomolecule analysis device comprises
a first electrode provided in the first liquid tank; and
a second electrode provided in each of the liquid tanks obtained by partitioning the second liquid tank.

8. A biomolecule analysis method for analyzing a biomolecule, the method comprising:
introducing the biomolecule into a liquid tank, the biomolecule having a first end portion connected to a control strand and a molecular motor, the control strand being bound to a primer on an upstream and having a spacer on a downstream, the liquid tank being disposed in contact with a thin film and containing an electrolyte solution, and the thin film having a nanopore;
applying a voltage to the liquid tank and introducing the biomolecule into the nanopore;
bringing the primer into contact with the molecular motor in the biomolecule introduced into the nanopore;
transporting the biomolecule in the nanopore by a synthetic reaction of the biomolecule after contact between the primer and the molecular motor; and
measuring a change of a current flowing in the nanopore during the transport.

9. The biomolecule analysis method according to claim 8, further comprising:
connecting an introductory strand for an introduction into the nanopore, to a second end portion of the biomolecule, the introductory strand having a double-stranded structure at at least an end portion on a side of the biomolecule, and having a single-stranded structure at an end portion on an opposite side of the biomolecule; and
introducing the single-stranded structure of the introductory strand into the nanopore and unzipping a double-stranded structure of the biomolecule to obtain a single-stranded structure.

10. The biomolecule analysis method according to claim 8, wherein the first end portion of the biomolecule is connected to the control strand and the molecular motor as a first molecular motor,
a second end portion of the biomolecule is connected to a second molecular motor different from the first molecular motor,
the first molecular motor is located between the spacer as a first spacer and the first end portion of the biomolecule, and
the second molecular motor is located between a second spacer and the second end portion of the biomolecule.

11. The biomolecule analysis method according to claim 10, further comprising:
dissociating a complementary strand of the biomolecule by the second molecular motor, wherein
the first molecular motor synthesizes the biomolecule after dissociation of the complementary strand by the second molecular motor, based on the primer.

12. The biomolecule analysis method according to claim 10, wherein the first molecular motor is a polymerase, and
the second molecular motor is a helicase.

13. The biomolecule analysis method according to claim 8, wherein a first stopper molecule and a second stopper molecule are further connected to both ends of the biomolecule, and
dimensions of the first and second stopper molecules are larger than a size of the nanopore.

14. A biomolecule analysis method for analyzing a biomolecule, the method comprising:
connecting a control strand to a first end portion of the biomolecule having a double-stranded structure and connecting an introductory strand to an end portion of the control strand on an opposite side of the biomolecule, the control strand including a molecular motor and a spacer between the molecular motor and the biomolecule, and the introductory strand having a double-stranded structure;
introducing the biomolecule into a liquid tank that is disposed in contact with a thin film and contains an electrolyte solution, the thin film having a nanopore;
applying a voltage to the liquid tank and introducing the introductory strand into the nanopore to dissociate the double-stranded structure of the introductory strand;
starting dissociation of the double-stranded structure of the biomolecule by bringing a complementary strand of the biomolecule into contact with the molecular motor after the dissociation of the double-stranded structure of the introductory strand;
transporting the biomolecule in the nanopore by a dissociation reaction of the biomolecule; and
measuring a change of a current flowing in the nanopore during the transport.
